(19) United States
(12) Patent Application Publication
(10) Pub. No.: US 2011/0276076 A1
(43) Pub. Date: Nov. 10, 2011

(54) Implantable Device With A Multi-Layer Envelope, And Corresponding Method Of Production

(75) Inventor: Pascal Paganon, Serezin Du Rhone (FR)

(73) Assignee: COMPAGNIE EUROPEENNE D'ETUDE ET DE RECHERCHE DE DISPOSITIFS POUR L'IMPLANTATION PAR LAPAROSCOPIE

(21) Appl. No.: 13/133,434

(22) PCT Filed: Dec. 9, 2009
(86) PCT No.: PCT/EP09/66674

§ 371 Date: Jul. 25, 2011

(30) Foreign Application Priority Data
Dec. 9, 2008 (FR) 0858411

(51) Int. Cl. A61M 29/00; C09J 5/02

(52) U.S. Cl. 606/192; 156/305; 156/306.9

(57) ABSTRACT

The invention relates to a medical or surgical device (1) implantable in a human or animal body, said device (1) comprising an envelope (3) consisting of a multi-layer complex, of which at least a first layer (4) is for the most part composed of a first flexible polymer, and of which at least a second layer (5) is for the most part composed of a second flexible polymer, the chemical composition of said first and second flexible polymers preventing the formation of chemical bonds between said first and second flexible polymers, and said envelope (3) being designed to be filled with an inflating fluid. Said device (1) comprises at least a third layer (6), which is designed to form chemical bonds with each of said first and second flexible polymers, in such a way as to join said first and second layers (4, 5) across substantially the entire surface thereof.

The present invention relates to the technical field of medical or surgical devices designed to be implanted in a human or animal body, particularly to implantable devices designed to receive an inflating fluid.

The invention particularly concerns implantable medical or surgical devices designed to be in contact with tissues, biological organs and/or body fluids that surround the devices during their implantation.

The invention more specifically concerns a medical or surgical device implantable in a human or animal body, said device comprising an envelope consisting of a multi-layer complex, of which at least a first layer is for the most part composed of a first flexible polymer, and of which at least a second layer is for the most part composed of a second flexible polymer, the chemical composition of said first and second flexible polymers preventing the formation of chemical bonds between said first and second flexible polymers, and said envelope being designed to be filled with an inflating fluid.

The invention also concerns a method for producing a medical or surgical device implantable in a human or animal body, said method comprising a step of producing an envelope consisting of a multi-layer complex, said envelope being designed to be filled with an inflating fluid, said step of producing the envelope comprising a first subsidiary step of producing at least a first layer for the most part composed of a first flexible polymer, and a second subsidiary step of producing at least a second layer for the most part composed of a second flexible polymer, the chemical composition of said first and second flexible polymers preventing the formation of chemical bonds between said first and second flexible polymers.

In cases of morbid obesity in humans, when conventional diets prove ineffective, it is often necessary to turn to medical treatments using implantable devices, for example an intragastric balloon, designed to reduce the volume of the stomach and/or to limit the flow of food. The practitioner generally introduces the intragastric balloon into the stomach without surgical intervention, under simple anesthesia, traditionally by endoscopy. Indeed, the balloon is most often inserted in a folded and non-inflated state through the natural passages of the mouth and then the esophagus as far as the inside of the patient's stomach.

Once positioned in the stomach, the intragastric balloon receives an inflating fluid, for example air or physiological saline, via a catheter connecting the balloon to the outside of the patient's body, in such a way that the balloon assumes a substantially spherical functional shape in which it has a therapeutic efficacy. Thus inflated, the balloon occupies a space in the stomach that can no longer be occupied by food, and this effectively limits the ingestion capacity of the patient. In addition, the presence of the balloon in the patient's stomach significantly slows down the flow of food, which also contributes to reducing the quantity of food ingested. Therefore, the use of an intragastric balloon, making it possible to reduce the space available for food in the stomach and helping to limit the speed of transit of food, quickly promotes a feeling of fullness in the patient who is eating the food and thus helps the patient lose weight.

The known intragastric balloons most often comprise a flexible pocket made of an elastomeric material having properties of flexibility and biocompatibility, so as to promote simple introduction of the balloon into the stomach and good tolerance of the balloon by the patient. These balloons in particular have sufficient flexibility to allow them to be inflated in a functional position and then deflated prior to being withdrawn at the end of treatment, without risk of bursting or of abrupt deflation of the balloon.

However, although they help patients slim, these known intragastric balloons nonetheless have a number of disadvantages.

Thus, intragastric balloons of this kind have a degree of porosity to the inflating fluid and generally have a tendency to gradually deflate after a certain period of use, which is all the more troublesome since the treatment of obesity generally has to continue for several months. This undesired gradual deflation proves especially problematic since it reduces the volume of the balloon, and this limits the efficacy of treatment and may sometimes force the practitioner to inflate the balloon again. A new intervention poses some risks, especially the risk of over-inflating the balloon, since the practitioner does not know precisely the volume of inflating fluid that has actually been evacuated from the balloon. This problem of untimely deflation is also accentuated in cases where these intragastric balloons are inflated with a gas. This is because gas generally passes through the wall of the balloon more easily than a liquid. However, using a gas, especially air, as the inflating fluid makes it possible to obtain a balloon that is more comfortable for the patient, since the balloon has a weight that is significantly lower, in the patient's stomach, than that of a balloon inflated with a liquid. It is therefore a real technical challenge to design a balloon which, on the one hand, is light and comfortable for the patient and which, on the other hand, ensures that the escape of inflating fluid remains limited and without consequence on the efficacy of the treatment.

Furthermore, the known earlier balloons may prove porous to the gastric fluid surrounding them, such that a greater or lesser amount of gastric fluid can then migrate into the balloon during the treatment. This presence of gastric liquid within the balloon can cause a number of problems, particularly that of degrading the constituent polymer of the balloon, especially because of the strong acidity of the gastric liquid. Such degradation of the interior of the balloon can reduce the hold of the balloon in the stomach and, for this reason, force the practitioner to remove the balloon from the patient.

The presence of gastric liquid within the balloon can also cause problems when the balloon is being removed. The reason for this is that, at the end of treatment, the practitioner deflates the balloon, generally with the aid of a needle, so as to extract all the inflating fluid from the balloon, such that the latter recovers its initial non-inflated state compatible with easy and atraumatic withdrawal from the body of the patient. In cases where a quantity of gastric fluid is present in the balloon, the content of the balloon becomes difficult to fully empty, and this prolongs and complicates the operation of removing the balloon and may in some cases cause removal of the balloon to be complicated, even traumatic.

With a view to overcoming the problem of leaktightness with respect to the inflating fluid, it is known to use a balloon consisting of two flexible pockets made of polymer, namely an inner pocket arranged inside an outer pocket. The two pockets are connected to each other in the area of a valve, which permits inflation of the inner pocket with the aid of an inflating fluid. The inner pocket thus acts as it were as an “air chamber”, and the increase in its volume, by virtue of inflating fluid being introduced, causes a concomitant increase in the volume of the second, outer pocket, so as to obtain an inflated balloon of substantially spherical shape.

Such a balloon comprising two flexible pockets made of polymer has the advantage of considerably reducing the porosity of the balloon, especially as it is necessary for the fluid to pass through two thicknesses of material in order to be able to enter or leave the balloon. Moreover, the two pockets can be produced from elastomeric materials of different natures, in such a way as to improve the leaktightness of the balloon while at the same time keeping it atraumatic. More precisely, the inner pocket is made of polyurethane, while the outer pocket is made of silicone. Polyurethane in fact has excellent leaktightness with respect to gases, but a certain weakness and a surface aspect that can prove aggressive to the biological tissues. This relative weakness and potentially traumatic characteristic are compensated by the outer pocket which, for its part, is made of silicone, a strong and atraumatic material.

This balloon with two pockets can solve all of the abovementioned problems of a balloon comprising a single flexible pocket, but it could be optimized. This is because the presence of polymers of different natures for each of the pockets, as dictated by the specific functions assigned to each of the pockets, can lead to problems when deflating and removing the balloon. The inner pocket may in fact have a tendency not to follow the deformation of the outer pocket and to have a behavior different from or even independent of the outer pocket. In this case, this may be reflected in a crumpling or wrinkling of the inner pocket while the outer pocket is positioned correctly in the axis of the esophagus during the removal of the balloon. This crumpling of the inner pocket tends to modify the volume occupied by the deflated balloon, and this makes the latter more traumatic with respect to the tissues and in some cases makes removal of the balloon more complicated and more traumatic.

It is additionally possible, in some circumstances, that a quantity of gastric fluid succeeds in migrating inside the balloon and remains trapped between the two pockets. The presence of gastric liquid between the two pockets can in some cases lead to difficulties at the moment of deflation. This is because complete emptying of the balloon takes longer and is more difficult when gastric fluid is present in the balloon. This presence of gastric liquid between the two pockets can sometimes prevent complete deflation of the balloon and therefore make the removal of the balloon from the patient's body more traumatic.

The objects of the invention are therefore to overcome the abovementioned disadvantages and to make available a novel implantable medical or surgical device which is atraumatic and leaktight with respect to fluids, which is easy to fit in place, and whose use is particularly simple and atraumatic for the patient.

Another object of the invention is to make available a novel implantable medical or surgical device made from materials that are inexpensive, well known, and particularly well tolerated by the organism.

Another object of the invention is to make available a novel implantable medical or surgical device whose simple design promotes its atraumatic behavior in the organism.

Another object of the invention is to make available a novel implantable medical or surgical device that has excellent leaktightness with respect to the fluid it contains and to the body fluids surrounding it, effectively and reliably throughout the duration of the treatment.

Another object of the invention is to make available a novel implantable medical or surgical device that can be easily removed from the patient's body, especially by limiting any trauma to the tissues.

Another object of the invention is to make available a novel implantable medical or surgical device whose dimensions give said device properties of leaktightness while at the same time keeping it atraumatic and flexible.

Another object of the invention is to make available a novel implantable medical or surgical device that can be used in the medical, surgical or cosmetic field, especially in the context of treatment against obesity, or as a medical device for collecting and/or delivering fluid.

Another object of the invention is to make available a novel method for producing an implantable medical or surgical device, said method comprising steps which are easy to implement and by which it is possible to obtain a novel implantable medical or surgical device which is atraumatic and leaktight with respect to fluids, which is easy to fit in place, and whose use is particularly simple and atraumatic for the patient.

Another object of the invention is to make available a novel method for producing a medical or surgical device, said method comprising a step that guarantees reliable and lasting leaktightness of said device with respect to the fluids.

Another object of the invention is to make available a novel method for producing a medical or surgical device, said method comprising an operation by which it is possible to ensure uniform leaktightness of said device with respect to the body fluid.

Another object of the invention is to make available a novel method for producing a medical or surgical device, said method comprising steps that are simple and easy to implement, and making it possible to obtain a device that is atraumatic and easy to introduce into and remove from the patient's body.

Another object of the invention is to make available a novel method for producing a medical or surgical device that can be used in the treatment of morbid obesity.

The objects of the invention are achieved with the aid of a medical or surgical device implantable in a human or animal body, said device comprising an envelope consisting of a multi-layer complex, of which at least a first layer is for the most part composed of a first flexible polymer, and of which at least a second layer is for the most part composed of a second flexible polymer, the chemical composition of said first and second flexible polymers preventing the formation of chemical bonds between said first and second flexible polymers, and said envelope being designed to be filled with an inflating fluid, said device being characterized in that it comprises at least a third layer, which is designed to form chemical bonds with each of said first and second flexible polymers, in such a way as to join said first and second layers across substantially the entire surface thereof.

The objects of the invention are also achieved with the aid of a method for producing a medical or surgical device implantable in a human or animal body, said method comprising a step of producing an envelope consisting of a multi-layer complex, said envelope being designed to be filled with an inflating fluid, said step of producing the envelope comprising a first subsidiary step of producing at least a first layer for the most part composed of a first flexible polymer, and a second subsidiary step of producing at least a second layer for the most part composed of a second flexible polymer, the chemical composition of said first and second flexible polymers preventing the formation of chemical bonds between said first and second flexible polymers, said method being characterized in that the production step comprises a third subsidiary step of producing at least a third layer, which is designed to form chemical bonds with each of said first and second flexible polymers, in such a way as to join said first and second layers across substantially the entire surface thereof.

Other objects and advantages of the invention will become clearer upon reading the following description and by referring to the attached drawings, which are given solely for illustrative purposes and do not limit the invention and in which:

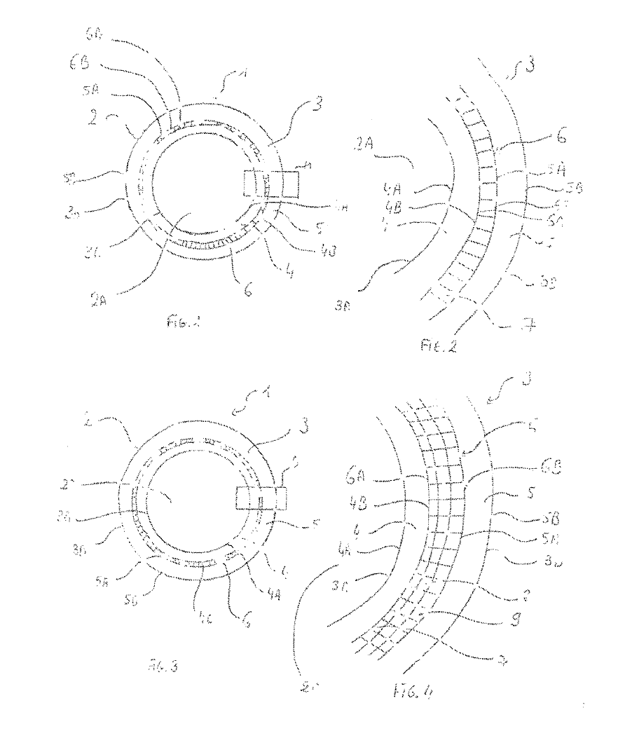

FIG. 1 shows, in a schematic cross section, an intragastric balloon according to a first embodiment of the invention,

FIG. 2 shows, in a schematic cross section, a detail A of the intragastric balloon from FIG. 1,

FIG. 3 shows, in a schematic cross section, an intragastric balloon according to a second embodiment of the invention,

FIG. 4 shows, in a schematic cross section, a detail B of the intragastric balloon from FIG. 3.

The present invention concerns a medical or surgical device 1 implantable in a human or animal body, that is to say a device 1 that can be introduced, implanted or inserted into the organism, especially in such a way that, once implanted, said device 1 is no longer directly accessible from outside the body. The device 1 is designed in particular to be in contact with body fluids during its introduction into the organism and once implanted. “Body fluid” is understood as a liquid or gas which is present in the body and with which the device 1 may be in contact, for example blood, mucus, gastric liquid, urine, or any other type of fluid present in the organism. Said device 1 is fitted in place during a surgical operation, involving local or general anesthesia, or during an endoscopy procedure or any other conventional surgical and/or medical operation. Preferably, the device 1 of the present invention is introduced into the human body by the natural routes, especially through the mouth and esophagus, during a simple medical operation that does not require surgical intervention.

It is entirely conceivable to use this device 1 for different applications, especially medical or surgical or cosmetic applications. Advantageously, the device 1 of the present invention constitutes one of the following devices: an intragastric balloon, a plastic surgery implant, a gastric band, or a device for injecting a fluid into and/or collecting a fluid from the body of the patient.

In the case where the device 1 constitutes a gastric band, it is designed to surround the stomach of a patient in order to reduce the passage diameter of the stomach for the treatment of obesity, that is to say it is designed to engage around the stomach, for example in the area of the cardia, in such a way that the flow of food and the quantity of food that can be ingested are greatly reduced, so as to help the patient slim.

In another embodiment, in which the device 1 of the invention constitutes a device for injecting and/or collecting fluid, it takes the form, for example, of an “implantable site” or of an “injection port” for repeated injection of medicaments. In cosmetic surgery, the device 1 constitutes a plastic surgery implant, for example a breast implant filled with silicone gel or saline solution. It is also conceivable, according to other embodiments of the present invention, that the device 1 constitutes an orthopedic or articular implant, for the replacement of a defective joint for example, or a urinary sphincter used in the treatment of urinary incontinence.

For the purpose of conciseness, the rest of the description will be confined to describing exclusively a preferred embodiment of the device 1 of the invention in which said device 1 is an intragastric balloon 2 designed to be implanted in the stomach of a patient in order to reduce the internal volume of the stomach for the treatment of morbid obesity, that is to say an intragastric balloon 2 designed to be positioned in the stomach of an obese patient in such a way as to play a part in the treatment of obesity.

The intragastric balloon 2 of the invention is designed to be introduced as naturally as possible into the stomach, in such a way as to reduce the volume of the stomach with the aim of helping the patient slim. The practitioner conventionally introduces the balloon 2 in a folded and non-inflated state through the mouth and then the esophagus until it reaches the stomach, where the balloon 2 is positioned at the desired location, the balloon 2 being connected permanently to the outside of the patient by way of a flexible tube made of biocompatible material, for example a catheter. Once in place in the stomach, the balloon 2 is filled with an inflating fluid by way of the catheter, such that the balloon 2 is in a substantially spherical shape, in which it has a therapeutic efficacy.

The balloon 2 comprises an envelope 3 consisting of a multi-layer complex, said envelope 3 being designed to serve as the single wall of said balloon 2. “Single wall” is understood as meaning that the balloon 2 has a single pocket separating the inside of the balloon 2 from the gastric environment. In other words, with its single wall forming the envelope 3, the balloon 2 has a behavior similar to that of a balloon comprising a single pocket, that is to say it has in particular a unified, unitary and homogeneous behavior during its deflation. This envelope 3 comprises an inner face 3A and an outer face 3B, the inner face 3A defining a substantially closed chamber 2A. The envelope 3 has properties allowing it, on the one hand, to form a balloon 2 of substantially spherical shape and, on the other hand, to ensure the secure positioning, resistance and therapeutic efficacy of the balloon 2 in the stomach of the patient.

The multi-layer complex of the invention comprises a combination of several layers forming an integral assembly, of which at least a first layer 4, comprising an inner face 4A and an outer face 4B, is for the most part composed of a first flexible polymer, and of which at least a second layer 5, comprising an inner face 5A and an outer face 5B, is for the most part composed of a second flexible polymer. All the layers of the envelope 3 are joined together across substantially the entire surface thereof. As is illustrated in the figures, the envelope 3 of the balloon 2 is therefore designed in the from of strata, of which at least two are of a different nature and are not chemically compatible, said strata forming layers stacked on top of one another and joined or glued to one another across substantially the entire surface thereof and encircling the chamber 2A, in such a way as to form a single wall for the balloon 2. In other words, the layers are superposed and joined together, across substantially the entire surface thereof, in a continuous manner and without any spaces between them, in such a way as to form a one-piece envelope 3 conferring on the balloon 2 a homogeneous behavior substantially identical to that of a single-pocket balloon. The presence of this single multi-layer envelope 3 has the advantage of preventing untimely penetration of gastric fluid between the two layers 4, 5. This is because, in the balloon 2 of the invention, there is no space available between the different constituent layers of the envelope 3, which eliminates the risks of gastric fluid stagnating between said layers and therefore makes it easier to deflate and withdraw the balloon 2.

The envelope 3 is preferably designed to be filled with an inflating fluid, which is contained in the chamber 2A delimited by said envelope 3. In other words, the balloon 2 is inflated until it reaches a functional volume effective in the context of a therapeutic treatment and substantially equal to 600 ml. By introducing inflating fluid into the envelope 3, in particular within the chamber 2A delimited by the envelope 3, it is therefore possible to bring the envelope 3, and more generally said balloon 2, from a low-volume configuration to an expanded configuration, in which said balloon 2 has its functional shape. Advantageously, the balloon 2 of the invention is inflated with a gas, for example air, in such a way as to obtain a balloon that is light and comfortable for the patient.

Since the balloon 2 is designed to pass from a folded configuration of low volume, during its introduction into the stomach, to an inflated configuration, it is advantageously made of flexible polymer permitting introduction, deployment, inflation, deflation and easy removal of the balloon 2. Advantageously, the first polymer of the envelope 3 is an elastomer, preferably polyurethane, and the second polymer of the envelope 3 is also an elastomer, preferably silicone. Thus, said envelope 3 is preferably for the most part composed of at least two layers 4, 5 of elastic polymers that are biocompatible and well tolerated by the organism. It is of course possible that the envelope 3 comprises more than two layers of elastomer and/or that the nature of the layers is modified, that is to say the outermost layer is made of polyurethane and the innermost layer is made of silicone.

Preferably, the first layer 4 of the envelope 3 is the innermost layer of the balloon 2 and is designed to be in contact with the inflating fluid, the second layer 5 of the envelope 3 being the outermost layer and being designed to be in contact with a body fluid, here the gastric liquid. When inflating fluid is introduced into the chamber 2A of the balloon 2, the first layer 4 deforms, by virtue of its elastic properties, and entrains in its deformation the second layer 5, which is attached to it. The first layer 4 is thus capable of expanding, when fluid is introduced, in order to assume a substantially spherical shape corresponding to the shape of the second layer 5 when inflating fluid is introduced, said second layer 5 advantageously having a memory for a substantially spherical shape. In addition, the first layer 4 has a certain degree of leaktightness with respect to the fluids possibly entering and/or leaving the balloon 2. This is because polyurethane is an elastomer that is less porous to fluids than silicone. Therefore, in addition to its abovementioned function of ensuring a spherical shape, the first layer 4 advantageously has the function of a natural barrier that limits the entry and/or escape of fluid into/from said balloon 2.

For its part, the second layer 5, preferably mainly composed of silicone, has the function of providing mechanical strength, of preventing trauma and of ensuring flexibility to promote good tolerance of the balloon 2 in the body. This is because silicone is a material that is perfectly well tolerated by the organism, limits the risks of infection, withstands the acidity of the environment in the stomach and is widely used in numerous medical or surgical applications inside the body. It additionally protects polyurethane from acid attack by the gastric liquid.

The combination of these two layers 4, 5 of elastomer in the envelope 3 therefore has the advantage of limiting the flow of fluids through said envelope 3, which greatly improves the efficacy of the balloon 2, in particular by avoiding untimely and premature deflation of the balloon, and by limiting the internal degradation of the balloon 2 by the particularly acidic gastric liquid that could infiltrate into the chamber 2A. This combination also makes it possible to obtain a flexible and atraumatic balloon 2.

Consequently, the balloon 2 of the invention comprises, on the one hand, at least a first and inner layer 4, of which the main function is to limit the transfer of fluids between the balloon 2 and the environment in the stomach, in both directions, and, on the other hand, at least a second and outer layer 5, of which the function is to make the balloon 2 both atraumatic and flexible. Thus, the constituent polymers of said layers 4, 5 are chosen for their properties in relation to these functions that said layers 4, 5 have to satisfy. Generally speaking, the properties of leaktightness are advantageously met by polymers of the polyurethane type, while the properties of flexibility and absence of trauma are preferably met by polymers of the silicone type.

However, these polymers are not chemically compatible with each other. This is because the chemical composition of said first and second flexible polymers, that is to say preferably polyurethane and silicone, of which said first and second layers 4, 5 respectively are for the most part composed, prevents the formation of chemical bonds between said first and second flexible polymers. In other words, because of their chemical incompatibility, the first and second polymers defined above, namely in particular silicone and polyurethane, cannot be made to adhere naturally, in particular in a spontaneous manner or with the use of conventional glues.

To overcome this problem, the balloon 2 also comprises at least a third layer 6, which comprises an inner face 6A and an outer face 6B and which is designed to form chemical bonds with each of said first and second flexible polymers, so as to join said first and second layers 4, 5 across substantially the entire surface thereof. Preferably, the third layer 6 is an intermediate layer positioned between the first layer 4 and the second layer 5 of the envelope 3. By virtue of its chemical nature in particular, this third layer 6 is capable of forming chemical bonds with each of the first and second layers, across the entire surface thereof. The chemical bonds are advantageously strong bonds, preferably covalent bonds, so as to allow the first and second layers 4, 5 to be joined firmly together.

In a particularly advantageous manner, and as is shown in FIG. 2, the third layer 6 of the envelope 3 comprises a single subsidiary layer 7 composed for the most part of at least a silicon-based biocompatible compound, preferably at least a silicon oxide and/or at least a silane and/or at least a siloxane and/or at least a carbosilicate compound. The first subsidiary layer 7 contributes to improving the leaktightness of the first layer 4, and more generally of the envelope 3, with respect to the gastric liquid, and this helps guarantee the stability of the balloon 2 throughout the duration of treatment, in particular by attenuating the porous character of the envelope 3 and by constituting an effective barrier for the latter.

In addition, these silicon-based compounds are mechanically strong and have sufficient flexibility to follow, without risk of fissure, the deformation of the envelope 3 during the inflation and deflation of the balloon 2. By way of example, the first subsidiary layer 7 can comprise a compound of formula SiCH and/or a silicon oxide of formula SiO₂ and/or a siloxane with a methyl group of formula SiO(CH₃)₃.

It is also conceivable that the first subsidiary layer of the third layer 6 comprises several of the compounds mentioned above, said compounds also being able to be arranged in the form of several strata stacked on top of one another, so as to improve the leaktightness of the envelope 3 and in particular of the first layer 4. In a first embodiment of the invention, the first subsidiary layer 7 of the third layer 6 of the envelope 3 is designed to form chemical bonds with the first layer 4 of the envelope 3 on the one hand and with the second layer 5 of the envelope 3 on the other hand. Thus, the first subsidiary layer 7 of the third layer 6 forms chemical bonds, preferably covalent bonds, with the polyurethane and the silicone, so as to join the first and second layers 4, 5 firmly together.

Preferably, the thickness of the first subsidiary layer of the third layer 6 of the envelope 3 is substantially between 50 nm and 2000 nm, and preferably substantially between 50 nm and 300 nm. Such a thickness is sufficient to ensure the leaktightness of the envelope 3 while at the same time keeping said envelope 3 flexible and atraumatic.

Moreover, the third layer 6 of the envelope 3 advantageously comprises a second subsidiary layer 8 composed for the most part of at least a biocompatible metal and/or of at least a biocompatible ceramic, preferably chosen from the following group: gold, silver, platinum, titanium, aluminum, aluminia, zirconia, titanium oxide. The composition of the second subsidiary layer 8 also contributes to ensuring the leaktightness of the envelope 3, especially by providing a barrier to the inflating fluid that may possibly escape from the balloon 2. In an advantageous embodiment, it will be preferable to use gold, which has properties of leaktightness, flexibility and malleability that are of particular interest in the context of the present invention. It is also conceivable that the third pocket 6 comprises only a second subsidiary layer 8, without silicon-based subsidiary layer, comprising one or more strata of metal and/or ceramic. Preferably, the thickness of the second subsidiary layer 8 of the third layer 6 of the envelope 3 is substantially between 50 nm and 2000 nm, and preferably substantially between 50 nm and 200 nm, preferably substantially equal to 100 nm.

Preferably, the third layer 6 of the envelope 3 is for the most part composed of at least a first subsidiary layer 7 and/or a second subsidiary layer 8, in such a way as to increase the leaktightness of the envelope 3, more generally of the balloon 2, both to the fluid that it contains and also to the body fluid. The third layer 6 also makes it easier to join the first and second layers 4, 5. Advantageously, the first subsidiary layer is deposited on the first layer 4 and acts as a fastener for the second subsidiary layer 8 on said first layer 4. This is because the silicon-based compound promotes the hold of the metal and/or of the ceramic constituting the second subsidiary layer 8 on the first layer 4. In addition, in the case where the third layer 6 comprises a metallic deposit in the area of its outermost subsidiary layer, use is made of adhesion primers, which are coated on the metallic subsidiary layer in such a way as to promote the formation of chemical bonds between the metal and the second layer 5 made of silicone.

In another advantageous embodiment of the present invention as shown in FIG. 4, the third layer 6 of the envelope 3 comprises at least a first subsidiary layer 7, at least a second subsidiary layer 8 and at least a third subsidiary layer 9, said first and third subsidiary layers 7, 9 being of substantially identical composition, the second subsidiary layer 8 being interposed between said first and third subsidiary layers 7, 9 in such a way that each of said first and third subsidiary layers 7, 9 is designed to form chemical bonds with the first and second layers 4, 5, respectively, of the envelope 3. Such a composition of the third layer 6 makes it possible to completely seal the balloon 2 and ensure that said first and second layers 4, 5 of the envelope 3 of the balloon 2 are joined across substantially the entire surface thereof. The third layer 6 thus advantageously acts as surface treatment of the first layer 4, in order to allow the latter to be joined to the second layer 5, preferably made of silicone.

Advantageously, the second layer 5 is coated with a silicone-based adhesive, said adhesive permitting the formation of chemical bonds, preferably of covalent bonds, between the third layer 6 and the second layer 5. Alternatively, it is possible to cause said third layer 6 to adhere to the second layer 5 by immersing the third layer 6 in a liquid elastomer, preferably silicone, designed to form the second layer 5 when cooled, the chemical bonds then forming naturally between said second and third layers 4, 5.

Preferably, all of the various layers described above make it possible to obtain an envelope 3 with a thickness substantially between 0.1 and 0.9 mm, advantageously substantially equal to 0.5 mm, preferably substantially less than 0.4 mm. The envelope therefore has the advantage, on the one hand, of providing a barrier to the inflating fluid, especially gas, and to the body fluid, for example the gastric liquid, and, on the other hand, of being sufficiently thin to preserve its elasticity and fold up suitably when deflated in order to facilitate the removal of the balloon 2 via the esophagus.

In a preferred embodiment of the present invention as shown in FIG. 4, the envelope 3 comprises the following layers, from the inside to the outside of the balloon 2:
A first layer 4 made of elastomer, preferably polyurethane, which delimits the chamber 2A of the balloon 2 and whose thickness is substantially less than 150 μm.
A first subsidiary layer 7 of a third layer 6 comprising at least a silicon-based biocompatible compound, which forms a homogeneous and amorphous subsidiary layer on the first layer 4 and whose thickness is substantially less than 0.3 μm.
A second subsidiary layer 8 of a third layer 6 comprising a metallic deposit based on at least a biocompatible metal and/or at least a biocompatible ceramic, preferably of gold. The second subsidiary layer 8 preferably forms a homogeneous and compact subsidiary layer which is integrally joined to the first subsidiary layer 7 and whose thickness is substantially less than 0.2 μm.
A third subsidiary layer 9 of a third layer 6 comprising at least a silicon-based biocompatible compound, which forms a homogeneous and amorphous subsidiary layer on the second subsidiary layer 8 and whose thickness is substantially less than 0.3 μm.
A second layer 5 made of elastomer, preferably of silicone, which is the outermost layer and is in contact with the gastric liquid and whose thickness is substantially less than 300 μm.

This superposed configuration of layers advantageously promotes the good hold of the first and second layers 4, 5 while at the same time giving the balloon 2 excellent leaktightness both to the inflating fluid, especially air, and also to the gastric liquid. Consequently, such a multi-layer complex forming the envelope 3 of the balloon 2 has the advantage of guaranteeing bidirectional leaktightness of the balloon 2, not only to the body fluid that may possibly enter the balloon 2, but also to the inflating fluid capable of escaping from said balloon 2. It is also conceivable to use a greater number of layers or subsidiary layers without departing from the scope of the present invention. It is especially possible that the inner face 4A of the first layer 4 and/or the outer face 5B of the second layer 5 is covered with one or more subsidiary layers comprising at least a silicon-based biocompatible compound and/or at least a biocompatible metal and/or at least a biocompatible ceramic, such as those described above.

The present invention also concerns, as an entirely separate invention, a medical or surgical device 1 implantable in a human or animal body, said device 1 comprising an envelope 3 which is designed, on the one hand, to be filled with an inflating fluid and, on the other hand, to be in contact with body fluids, said envelope 3 consisting of a multi-layer complex, of which at least a first layer 4 is for the most part composed of a first polymer having properties of leaktightness to the inflating fluids and/or to the body fluids, and at least a second layer 5 is for the most part composed of a second polymer having properties of flexibility and absence of trauma, said device 1 being characterized in that it comprises at least a third layer 6, which is designed to form chemical bonds with each of said first and second polymers, in such a way as to join said first and second layers 4, 5 across substantially the entire surface thereof. The first and second polymers are preferably elastomers such as those described above, especially polyurethane and silicone respectively, which are not chemically compatible spontaneously or with the use of conventional adhesives.

The balloon 2 of the invention thus has the advantage, on the one hand, of being leaktight to the entry and escape of fluid, especially because of the presence of the first layer of polyurethane and the intermediate third layer 6 and, on the other hand, of being atraumatic and well tolerated by virtue in particular of its outer second layer 5 made of silicone. The multi-layer configuration of the envelope 3, in particular with the presence of a third layer 6 by means of which said first and second layers 4, 5, which are not naturally chemically compatible, are designed to be joined across substantially the entire surface thereof, makes it possible to give the balloon 2 the behavior of a single-pocket balloon. Indeed, the presence of the envelope 3, forming a contiguous single wall of which all the layers are perfectly joined continuously across substantially the entire surface thereof, permits simple and atraumatic removal of the balloon 2 via the esophagus without the envelope 3 creasing. In addition, the presence of a single envelope 3 avoids any risk of gastric liquid entering and stagnating between the layers of the balloon 2.

The present invention also concerns a method for producing a medical or surgical device implantable in a human or animal body. The following description will preferably set out a method for producing an intragastric balloon for the treatment of obesity, said balloon being designed to be implanted in the stomach of a patient in order to reduce the internal volume of the stomach. It is of course conceivable, without departing from the scope of the present invention, that the method can be used for producing other types of implants and can also be, for example, a method for producing a gastric band for the treatment of obesity, a urinary sphincter for the treatment of urinary incontinence, a plastic surgery implant or a device for injecting fluid into and/or collecting fluid from the body of the patient.

Said method comprises a step of producing an envelope 3 consisting of a multi-layer complex, said envelope 3 being designed to be filled with an inflating fluid. The step of producing the envelope 3 comprises a first subsidiary step of producing at least a first layer 4 for the most part composed of a first flexible polymer, preferably an elastomer, for example polyurethane.

Advantageously, the first layer 4 is produced by joining two sheets of polyurethane by high-frequency welding and is designed to contain the inflating fluid, preferably a gas.

This method also comprises a second subsidiary step of producing at least a second layer 5 for the most part composed of a second flexible polymer, preferably an elastomer, for example silicone. This second subsidiary step advantageously comprises an operation of molding the second layer 5 in order to obtain a second layer 5 of substantially spherical shape and with a shape memory, that is to say that the second layer 5, after molding, retains a memory of a substantially spherical shape.

The chemical composition of said first and second flexible polymers prevents the formation of chemical bonds between said first and second flexible polymers, especially between polyurethane and silicone, the latter being known to be non-adhesive.

Before said first and second layers 4, 5 are joined together, the production step of the method according to the invention comprises a third subsidiary step of producing at least a third layer 6, which is designed to form chemical bonds with each of said first and second flexible polymers, in such a way as to join said first and second layers 4, 5 across substantially the entire surface thereof. Advantageously, the third subsidiary step of production is performed after the first subsidiary step of producing the first layer 4, prior to the second subsidiary step of producing the second layer 5. The third subsidiary step of production preferably comprises a first operation of depositing a first subsidiary layer 7 for the most part composed of at least a silicon-based biocompatible compound, preferably a silicon oxide and/or a silane and/or a siloxane and/or a carbosilicate compound.

Preferably, the first operation of deposition comprises a step of providing leaktightness and is carried out by cold plasma deposition corresponding in particular to a cold plasma-assisted chemical vapor deposition (PACVD). This technique involves delivering one or more gases, for example hexamethyldisilane (HMDS) and/or tetramethylsilane (TMS) and/or hexamethyldisiloxane (HMDSO) and ionizing said gas or gases. Preferably, the vapor-phase deposition operation comprises grafting the silicon-based compound on the first layer 4, said grafting making it possible to form covalent bonds, on the one hand between the molecules of said silicon-based compound themselves and, on the other hand, between the molecules of said silicon-based compound and the molecules of said first layer 4 of the balloon 2.

Preferably, the third subsidiary step of production also comprises a second operation of depositing a second subsidiary layer 8 for the most part composed of at least a biocompatible metal and/or at least a biocompatible ceramic advantageously chosen from the following group: gold, silver, platinum, titanium, aluminum, zirconium, titanium oxide. Preferably, the second subsidiary layer 8, preferably for the most part composed of gold, is also deposited by cold plasma deposition, and the first subsidiary layer 7 deposited beforehand on the first layer 4 advantageously forms a subsidiary layer for effectively holding the second subsidiary layer 8 on the first layer 4. In other words, said first subsidiary layer 7 thus constitutes an effective and lasting adhesion interface between the metal and the polyurethane constituting the first layer 4.

It is also conceivable, without departing from the scope of the present invention, that the third subsidiary step of production also comprises a third operation of deposition of a third subsidiary layer 9 for the most part composed of at least a silicon-based biocompatible compound as defined above for the first subsidiary layer 7. The operation of deposition of the third subsidiary layer 9 is preferably carried out by a technique of cold plasma deposition substantially identical to that mentioned above.

Cold plasma deposition makes it possible, on the one hand, to obtain homogeneous subsidiary layers 7, 8, 9 of constant thickness and, on the other hand, to form solid bonds between the molecules of deposited silicon-based compounds, and between the molecules of silicon-based compounds and said first and/or second layers 4, 5. This technique makes it possible in particular to obtain crosslinking between the molecules of the first and/or third subsidiary layers 7, 9 between themselves and also between the molecules of the first and/or third subsidiary layers 7, 9 and the molecules of the first layer 4 and/or of the second layer 5. This is because the molecules are bonded to one another monomer by monomer, which helps cover the pores of the constituent polymer of the first layer 4 and/or of the second layer 5 and thus improves the leaktightness of the balloon 2.

Alternatively, any other method permitting the deposition of the subsidiary layers 7, 8, 9 of the envelope 3 may be envisioned, for example chemical vapor deposition (CVD) for deposition on materials that are not thermosensitive.

The production of the third layer 6 by the PACVD technique advantageously permits continuous deposition of several alternative strata of silicon-based compounds and/or of metal and/or of ceramic one after another, without changing or stopping the machine. This technique also avoids the columnar structures of metal that may possibly form. Preferably, the cold plasma deposition is performed when the first layer 4 is in an expanded configuration with a volume at least equal to the volume that the balloon 2 will occupy in the patient's stomach, the aim of this being to avoid possible subsequent risks of fissuring of the third layer 6 during inflation of the balloon 2. Moreover, the balloon 2 is preferably moved in rotation during the cold vapor deposition of the third layer 6, so as to promote uniform deposition of constant thickness.

Preferably, the production step additionally comprises an operation in which the first layer 4, previously covered by the third layer 6, is glued to the second layer 5, the gluing comprising an operation of coating said second layer 5 and/or the third layer 6 with a polymer glue that forms chemical bonds between the third layer 6 and the second layer 5, in order to join said first and second layers 4, 5 together. In this embodiment, the gluing operation takes place after the subsidiary steps of producing the first and third layers 4, 6 of the envelope 3. During the gluing operation, silicone adhesive is preferably deposited on the inner face 5A of the second layer 5 made of silicone, which is then joined to the first layer 4 made of polyurethane, the silicone adhesive permitting the formation of chemical bonds, especially covalent bonds of the Si—SiO type, between the third layer 6 and the second layer 5 in order to join said first and second layers 4, 5 together. It is also conceivable that the polymer glue is deposited on any one of the subsidiary layers 7, 8 or 9 of the third layer 6 when said subsidiary layer is in contact with said second layer 5.

Alternatively, it is also possible that the production step comprises an operation in which the first layer 4, previously covered by the third layer 6, is immersed in a liquid polymer that forms chemical bonds, in particular covalent bonds of the Si—SiO type, with the third layer 6, in order to join said first and second layers 4, 5 together. In this advantageous embodiment of the envelope 3 of the balloon 2, a first layer 4 is produced and is covered, according to the method described above, with a third layer 6. The envelope 3 is then immersed in a liquid polymer, preferably in liquid silicone, the third layer 6 then forming covalent bonds with the silicon atoms of the liquid silicone, so as to promote the formation and hold of the second layer 5, made of silicone, on the first layer 4. This immersion operation makes it possible to obtain a second layer 5 having a thickness of substantially less than 150 μm, thereby resulting in an intragastric balloon 2 comprising an envelope 3 with a thickness substantially less than the thickness of the two pockets of the earlier balloons.

By using the method according to the present invention, it is possible to obtain an intragastric balloon 2 comprising an envelope 3 which forms a single wall and of which all the layers are perfectly joined across substantially the entire surface thereof. This method therefore results in a balloon 2 that is leaktight to fluids, has a single wall, is easy to use and is atraumatic. In particular, such a method guarantees the production of a balloon 2 which does not risk creasing during its removal from the stomach and which, during use, has an overall behavior substantially identical to that of a balloon 2 comprising a single pocket.

1. A medical or surgical device implantable in a human or animal body, said device comprising:
an envelope consisting of a multi-layer complex, of which at least a first layer is for the most part composed of a first flexible polymer, and of which at least a second layer is for the most part composed of a second flexible polymer, the chemical composition of said first and second flexible polymers preventing the formation of chemical bonds between said first and second flexible polymers, and said envelope being designed to be filled with an inflating fluid, said device being characterized in that it comprises at least a third layer, which is designed to form chemical bonds with each of said first and second flexible polymers, in such a way as to join said first and second layers across substantially the entire surface thereof.

2-22. (canceled)

23. The device as claimed in claim 1, characterized in that the first polymer of the envelope is an elastomer, preferably polyurethane.

24. The device as claimed in claim 1, characterized in that the second polymer of the envelope is an elastomer, preferably silicone.

25. The device as claimed in claim 1, characterized in that the first layer of the envelope is designed to be in contact with the inflating fluid, the second layer of the envelope being designed to be in contact with a body fluid.

26. The device as claimed in claim 1, characterized in that the third layer is positioned between the first layer and the second layer of the envelope.

27. The device as claimed in claim 1, characterized in that the third layer of the envelope comprises a first subsidiary layer composed for the most part of at least a silicon-based biocompatible compound.

28. The device as claimed claim 1, characterized in that the third layer of the envelope comprises a second subsidiary layer composed for the most part of at least a biocompatible metal and/or of at least a biocompatible ceramic.

29. The device (1) as claimed in claim 27, characterized in that the third layer of the envelope is for the most part composed of at least a first subsidiary layer and/or a second subsidiary layer, in such a way as to increase the leaktightness of the envelope with respect both to the fluid that it contains and also to the body fluid.

30. The device as claimed claim 27, characterized in that the first subsidiary layer of the third layer of the envelope is designed to form chemical bonds with the first layer of the envelope on the one hand and with the second layer of the envelope on the other hand.

31. The device as claimed in claim 27, characterized in that the third layer of the envelope comprises at least a first subsidiary layer, at least a second subsidiary layer and at least a third subsidiary layer, said first and third subsidiary layers being of substantially identical composition, the second subsidiary layer being interposed between said first and third subsidiary layers, in such a way that each of said first and third subsidiary layers is designed to form chemical bonds with the first and second layers of the envelope respectively.

32. The device as claimed in claim 1, characterized in that the chemical bonds are strong bonds, preferably covalent bonds, so as to allow the first and second layers to be firmly connected to each other.

33. The device as claimed in claim 27, characterized in that the thickness of the first subsidiary layer of the third layer of the envelope is substantially between 50 nm and 2000 nm, and preferably substantially between 50 nm and 300 nm.

34. The device as claimed in claim 28, characterized in that the thickness of the second subsidiary layer (8) of the third layer (6) of the envelope (3) is substantially between 50 nm and 2000 nm, and preferably substantially between 50 nm and 200 nm.

35. The device (1) as claimed in claim 1, characterized in that it constitutes one of the following devices: an intragastric balloon designed to be implanted in the stomach of a patient in order to reduce the internal volume of the stomach for the treatment of morbid obesity, a plastic surgery implant, a gastric band designed to surround the stomach of a patient in order to reduce the passage diameter of the stomach for the treatment of obesity, a device for injecting fluid into and/or collecting fluid from the body of the patient.

36. The device as claimed in claim 1, characterized in that, by introducing inflating fluid into the envelope, it is possible to bring the device from a low-volume configuration to an expanded configuration in which said device has its functional shape.

37. A method for producing a device implantable in a body, said method comprising:
producing an envelope consisting of a multi-layer complex, said envelope being designed to be filled with an inflating fluid, said producing the envelope comprising:
producing at least a first layer for the most part composed of a first flexible polymer; and
producing at least a second layer for the most part composed of a second flexible polymer, the chemical composition of said first and second flexible polymers preventing the formation of chemical bonds between said first and second flexible polymers,
producing at least a third layer, which is designed to form chemical bonds with each of said first and second flexible polymers, in such a way as to join said first and second layers across substantially the entire surface thereof.

38. The method as claimed in claim 37, characterized in that the third subsidiary step of production is performed after the first subsidiary step of producing the first layer, prior to the second subsidiary step of producing the second layer.

39. The method as claimed in claim 37, characterized in that the third subsidiary step of production comprises a first operation of depositing a first subsidiary layer for the most part composed of at least a silicon-based biocompatible compound.

40. The method as claimed in claim 37, characterized in that the third subsidiary step of production comprises a second operation of depositing a second subsidiary layer for the most part composed of at least a biocompatible metal and/or at least a biocompatible ceramic.

41. The method as claimed in claim 37, characterized in that the production step comprises an operation in which the first layer, previously covered by the third layer, is glued to the second layer, the gluing comprising an operation of coating said second layer and/or the third layer with a polymer glue that forms chemical bonds between the third layer and the second layer, in order to join said first and second layers together.

42. The method as claimed in claim 37 characterized in that the production step comprises an operation in which the first layer, previously covered by the third layer, is immersed in a liquid polymer that forms chemical bonds with the third layer, in order to join said first and second layers together.

43. The method of claim 37 wherein the device is an intragastric balloon for the treatment of obesity, said balloon being designed to be implanted in the stomach of a patient in order to reduce the internal volume of the stomach.
